(19) United States
(12) Patent Application Publication
(10) Pub. No.: US 2011/0293753 A1
(43) Pub. Date: Dec. 1, 2011

(54) Tocotrienol Compositions

(76) Inventors: Louis Bellafiore, Wilmette, IL (US); James A. Radosevich, Belvidere, IL (US); Ed Glicken, Wilmette, IL (US)

(21) Appl. No.: 12/790,292

(22) Filed: May 28, 2010

(51) Int. Cl. A61K 36/889; A61P 29/00; A61K 36/9066; A61P 31/10; A61P 33/00; A61P 39/00; A61P 35/00; A61P 31/04

(52) U.S. Cl. 424/727; 424/756

(57) ABSTRACT

Compositional formulations are provided that comprise at least one tocotrienol or a derivative thereof, in combination with compounds derived from plant extracts. The compositions can be provided in a functionally acceptable carrier, or separately in a combined regimen. In some examples, the compositions can be effective for reducing, preventing or treating medical conditions including, for example, benign tissue growths, pre-cancerous lesions, cancer, inflammations, viral infections, bacterial infections, fungal infections, parasitic infections, impaired bodily function, or cell and tissue damage due to trauma, cell and/or tissue injury from stroke events, cell and/or tissue damage from ischemic events. The mechanism of action for cancer prevention and therapy includes telomerase and/or angiogenesis inhibition.

### BACKGROUND

Vitamin E is a generic name for a family of four compounds (forms) of tocopherols, four compounds of tocotrienols, four compounds of tocodienols and four compounds of tocomonoenols. All sixteen compounds have a chromanol ring structure and a side chain. There are four tocopherol forms (alpha, beta, delta, and gamma) with a fully saturated side chain; and four tocotrienol forms (alpha, beta, delta, and gamma) having unsaturated side chains with double bonds at the 3′, 7′, and 11′ positions in the side chain. In addition there are four tocodienol and tocomonoenol forms with two double bonds and one double bond respectively in the side chains. The four forms of each of the tocopherols, tocotrienols, tocodienols and tocomonoenols differ from each other in the number and position of methyl groups in the aromatic chromanol ring.

Vitamin E has been recognized to provide health benefits, at least in part due to its functionality as an antioxidant. In particular, Tocotrienols have three double bonds in their side chains, rather than being saturated. Tocotrienols can provide stronger antioxidant effects than tocopherols due to their unsaturated side chains. Tocomonoenols and tocodienols are intermediate in saturation and antioxidant effect between tocopherols and tocotrienols and have been identified as. Tocotrienols have been identified in nature in sources such as rice bran, palm fruit, annatto plant seeds, and certain forms of algae. It is expected that new sources will be discovered, created or optimized through breeding, as well as, genetically engineered cell lines including but not limited to mammalian, non-mammalian animal cells, plant cells, other multi-cellular and single cell organism such as algae, fungi, and bacteria. Genetically engineered plants, mammalian and non-mammalian animals could also be used to produce tocotrienols. Natural sources of tocotrienols generally include one or more tocotrienols and one or more tocopherols.

### BRIEF SUMMARY

The current invention involves the use of tocorienols or their derivatives, as the primary preventative or therapeutic agent, combined in synergistic formulations with secondary compounds that are preferably found in nature.

In one aspect, a compositional dosage for daily administration to a subject is provided that includes at least one tocotrienol and at least one secondary component. The at least one tocotrienol can be selected from the group consisting of gamma tocotrienol or a derivative of gamma tocotrienol, delta tocotrienol or a derivative of delta tocotrienol, beta tocotrienol or a derivative of beta tocotrienol, and combinations thereof. The at least one tocotrienol can be present in an amount from about 10 mg to about 2 g and at the least one secondary component can be present in an amount of up to about 5 g. The weight ratio of at least one tocotrienol to the at least one secondary component can be from about 1:10 to about 10:1.

In some examples, the at least one secondary component can be derived from a natural source. In at least one example, a mixture of secondary components is provided. In such an example, the mixture of secondary components can include at least turmeric extract compounds and fermented noni juice compounds.

In another aspect, the compositional dosage can include a dispersion medium. In such an example, the compositional dosage can be a suspension or a colloid of the at least one tocotrienol and the at least one secondary component in a dispersion medium.

### DETAILED DESCRIPTION

The present technology relates to compositional dosages for daily administration to a subject, where the compositional dosages contain tocotrienol and at least one secondary component. The compositional dosages can be any suitable type of formulation, including, for example formulations for pharmaceutical, nutriceutical, or veterinary purposes. The compositional dosages described herein preferably comprise a therapeutically effective amount of the at least one tocotrienol and the at least one secondary component, and can be utilized to prevent or treat one or more medical conditions, including, for example, benign tissue growths, pre-cancerous lesions, cancer, inflammations, viral infections, bacterial infections, fungal infections, parasitic infections, impaired bodily function, or cell and tissue damage due to trauma, cell and/or tissue injury from stroke events, cell and/or tissue damage from ischemic events by a number of possible delivery routes. This includes the specific use of these compositional dosages as telomerase and/or angiogenesis inhibitors. The compositional dosages described herein can be administered to subjects as part of a treatment regimen, either alone or in combination with other methods of treating these medical conditions, including, for example, surgery, radiation and chemotherapy.

The total compositional dosage can be administered to the subject in one or more units during a on a daily bases, which means in any given twenty-four hour period. Additionally, the tocotrienol component and the at least on secondary component can be formulated together or administered separately. In one example, the total compositional dosage, or individual portions thereof, can be contained in a delivery system adapted for any suitable type of administration, including but not limited to oral, topical, intraocular, parenteral, intranasal, intravenous, intramuscular, or subcutaneous. In some examples for topical administration, the delivery system can be an ointment or cream, or can be delivered using drug patch technology. In some examples for oral administration, the delivery system can be capsules such as gelatin capsules, tablets, liquid solutions, suspensions, or elixirs. In other example, the total compositional dosage, or individual portions thereof, can be incorporated into nutritional drinks or foods, including but not limited to butter, peanut butter, cereal, nut coatings, margarine, meat and processed meats, soups, purees, and the like. Incorporation of the compositional dosages into nutritional food or drinks can be accomplished by any suitable means, including by conventional procedures that maintain vitamin efficacy such as low heat, inert atmosphere blending.

The subject can be a human of any suitable age, and is preferably a human male or female that has reached adulthood. Alternatively the subject can be an animal, including mammals and non-mammals, and can, for example, be an animal commonly kept by people as house pets, such as a dog, a cat, a bird, or a fish.

Compositional dosages of the present technology can include at least one tocotrienol in an amount from about 10 mg to about 2 g, preferably in an amount from about 50 mg to about 1 g. Tocotrienols generally have the following chemical structure:

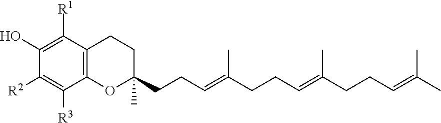

In alpha tocotrienol, R¹ is Me, R² is Me, and R³ is also Me. In beta tocotrienol, R¹ is Me, R² is H, and R³ is Me. In gamma tocotrienol, R¹ is Me, R² is Me, and R³ is H. In delta tocotrienol, R¹ is Me, R² is H, and R³ is also H.

Tocomonoenols and Tocodienols are similar in structure to the Tocotrienols above, with the difference being the degree of saturation, with the Tocomonenols having one double bond in the tail chain and the Tocodienols having two double bonds in the tail chain, as compared with three double bonds in the Tocotrienols and none in Tocopherols.

Tocotrienols useful in the present compositions can include any tocotrienol or tocotrienol derivative. Derivatives of tocotrienols can be of any suitable type, and preferably are of a type that increase the adsorption rate or the duration of effect of the component. Accordingly, the “at least one tocotrienol” discussed herein can include one or more forms of tocotrienol, including alpha tocotrienol, beta tocotrienol, delta tocotrienol and gamma tocotrienol, a derivative of any form of tocotrienol, or a combination of forms of tocotrienol and/or derivatives of any form of tocotrienol. In some examples, the compositional dosages described herein include at least gamma tocotrienol or a derivative of gamma tocotrienol. In other examples, compositional dosages can include at least delta tocotrienol or a derivative of delta tocotrienol. In still further examples, the compositional dosages described herein include both at least gamma tocotrienol or a derivative of gamma tocotrienol, and at least delta tocotrienol or a derivative of delta tocotrienol.

In some examples, the compositional dosages are tocopherol-free or substantially tocopherol-free. Substantially tocopherol-free refers to compositional dosages containing tocopherols in an amount about 5% by weight of the compositional dosage or less. Such tocopherol-free compositional dosages can contain tocopherols in an amount from about 0% by weight to about 5% by weight by weight of the compositional dosage, preferably from about 0% by weight to about 2% by weight of the compositional dosage. In some examples, compositional dosages contain tocopherols in amounts of about 0.5% by weight, about 1% by weight, about 1.5% by weight, about 2% by weight, about 2.5% by weight, about 3% by weight, about 3.5% by weight, about 4% by weight, or about 4.5% by weight of the compositional dosage.

In order to produce compositional dosages that include specific types of tocotrienols, and those that are substantially tocopherol-free, it may be desirable to separate and isolate tocotrienols that can be obtained from various sources. One example of isolating desired tocotrienols is described in U.S. Pat. No. 6,395,915 to Bellafiore et al., the disclosure of which is hereby incorporated by reference in its entirety. In some examples, compositional dosages can include gamma tocotrienol that has been isolated from a tocotrienol source, and can also include a combination of tocotrienols that have been isolated from a tocotrienol source.

Compositional dosages of the present technology can also include at least one secondary component, which can be present in an amount of up to about 5 g. The amount of the at least one secondary component can be selected so that the weight ratio of at least one tocotrienol to the at least one secondary component is from about 1:10 to about 10:1.

In one preferred example, the at least one secondary component is derived from a natural source. Without being bound by any particular theory, it is believed that secondary components derived from natural sources may contain analogs or other naturally occurring compounds that can provide a synergistic or direct effect in combination with the other components of the compositional dosage. However, extracts of natural products, such as for example tumeric extracts, may vary widely in strength and impurity levels, including naturally occurring impurities and isomers, pesticides, herbicides and heavy metals, depending on the type of the source and the extraction techniques employed, and the final concentration process. Examples of extraction techniques can include solvent extraction, supercritical fluid extraction or distillation. Accordingly, in preparing compositional dosages of the present technology, it is desirable that the purity of each secondary component be monitored and tested for quality control, and that the dosages of each secondary component be adjusted to ensure that desired amount is present in the desired compositional dosage.

Synthetic compounds can be utilized as primary (i.e. tocotrienol) or secondary components in some examples. However, the synthesis process can result in the production of by-products or the inclusion of impurities that can have an effect that is opposite or in competition with that of the desired component. One well known historical example is the production of the thalidomide which produced optical isomers (enantiomers) of the desired compound of which the ‘S’ enantiomer was tetragenic but the ‘R’ isomer was an effective sedative. Accordingly it is preferred that synthetic compounds be substantially free of potentially toxic, antagonistic or otherwise undesired by-products or impurities, and that they be demonstrated to have equivalent adsorption, distribution, metabolism, excretion, and toxicity profiles as components derived from natural sources, and preferably also an equivalent bioequivalency.

In some examples, at least one secondary component can be turmeric extract compounds, beta-carotene, saw palmetto extract compounds, fermented noni juice compounds, L-ascorbic acid, aloe vera compounds, Solanum Dulcamara extract compounds, Celastrol, Garcinia mangostana L. (Guttiferae) pericarp extract compounds, rutin, quercetin, ginko bilboa extract compounds, ocimum sanctum extract compounds, rosemary extract compounds, blueberry extract compounds, Withania somnifera Dunal extract compounds, Rhodiola extract compounds, Schizandra berry extract compounds, or a combination thereof. As discussed above, any secondary component can be derived from a natural source, or can be synthetic. It should be understood that the term “extract compounds” refers to any and all compounds that can be derived from an extract of a given natural source, and that synthetic versions of such compounds are also encompassed.

Tumeric extract compounds can include curcumin, desmethoxycurcumin and bis-desmethoxycurcumin. Curcumin is the principal curcuminoid found in the spice tumeric. Curcumin generally has the following chemical structure:

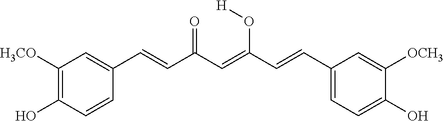

Curcumin can exist in at least two tautomeric forms, the keto and the enol. The keto form generally has the following chemical structure:

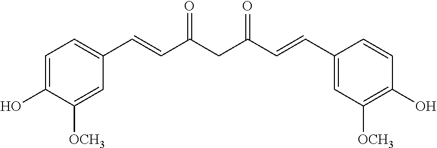

The enol form of Curcumin generally has the following chemical structure:

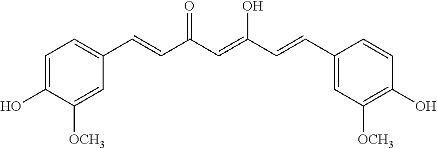

Beta-carotene is believed to have both antioxidant and potential anti-cancer activity. Studies that have used synthetic source beta-carotene indicated beta-carotene may cause cancer however, this could be related to impurities present in the synthetic source materials such as undesired reaction side products or trace levels of unconsumed reactants themselves.

L-ascorbic acid, which can be found in several natural sources, may provide increased adsorption or utilization of the other formulation compounds as well as providing its own benefits.

Saw palmetto, also known as serenoa repens, sabal serrulatum, and other alternative names, is the sole species currently classified in the genus Serenoa, and is a natural herb that has been shown to be an effective antiandrogen.

Fermented noni juice compounds can be derived from Morinda citrifolia, which is commonly known as great morinda, Indian mulberry, Nunaakai, Dog Dumpling, Mengkudu, beach mulberry, Tahitian noni, noni, vomit fruit, and cheese fruit. Morinda city folia is a tree in the coffee family, Rubiacea. Noni juice can contain a number of phytochemicals, including lignans, oligo- and polysaccharides, flavonoids, iridoids, fatty acids, scopoletin, catechin, beta-sitoserol, damnacanthal, and alkaloids. Fermented noni juice compounds can be utilized to treat a wide variety of medical conditions, including but not limited to arthritis, atherosclerosis, benign lesions, bladder infections, boils, bowel conditions, burns, cancer, chronic fatigue syndrome, circulatory weakness, colds, cold sores, constipation, diabetes, drug addiction, eye inflammation, fever, fractures, gastric ulcers, gingivitis, headaches, heart disease, hypertension, improved digestion, immune weakness, indigestion, kidney disease, malaria, menstrual cramps, menstrual disorders, mouth sores, pre-cancerous lesions, respiratory disorders, ringworm, sinusitis, skin inflammation, sprains, strokes, thrush, wounds, and can they can also act as an anticoagulant.

Solanum Dulcamara includes trailing nightshade, bittersweet, trailing bittersweet, climbing nightshade, blue bindweed, bitter nightshade, fellenwort, dogwood, woody nightshade, poisonflower, poisonberry, snakeberry, and scarlet berry.

Celastrol is a quinone methide triterpene present in Celastraceae plants and is known to have multitude arrays of pharmacological activities. For example, it has been used for the treatment of breathing problems in people with asthma because it is a long-acting bronchodilator, acting to help keep the airways open. It has also been used in the treatment of autoimmune diseases, chronic inflammation, and neurodegenerative disease. It has also been shown to inhibit cancer cell proliferation and induce leukemic cell death. Studies have also shown that Celastrol has pharmacological activities associated with anti-infective properties One common source of Celastrol is found in Tripterygium wilfordii Hook F, which is an ivy-like vine. Celestrol generally has the following chemical structure:

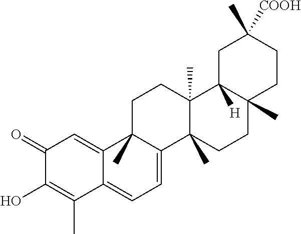

Garcinia mangostana L. (Guttiferae) pericarp extract compounds can include xanthones such as mangostinone, alpha-mangostin, beta-mangostin, gamma-mangostin, gartanin, garcinone E, 1,5-dihydroxy-2-(3-methylbut-2-enyl)-3-methoxy xanthone, and 1,7-dihydroxy-2-(3-methylbut-2-enyl)-3-methoxy xanthone. Garcinia mangostana L. (Guttiferae) pericarp extract compounds can also include lignans, oligo- and polysaccharides, flavonoids, iridoids, fatty acids, scopoletin, catechin, beta-sitoserol, damnacanthal, and alkaloids. Some examples of the chemical structures of certain Garcinia mangostana L. (Guttiferae) pericarp extract compounds are provided here for reference. For example, Alpha-mangosteen generally has the following chemical structure:

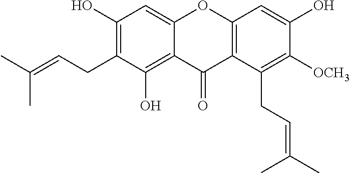

Beta-mangosteen generally has the following chemical structure:

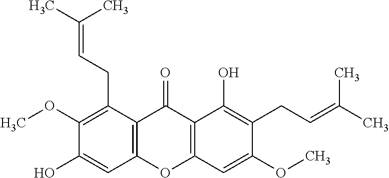

Gamme-mangosteen generally has the following chemical structure:

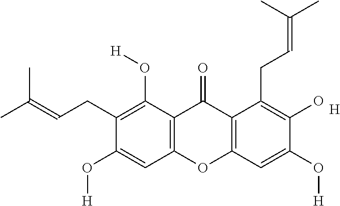

Garcinone D generally has the following chemical structure:

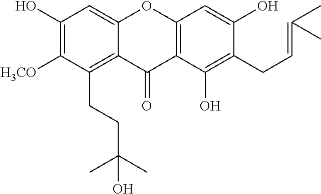

Garcinone C generally has the following chemical structure:

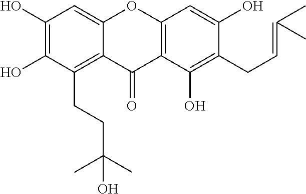

Gartanin generally has the following chemical structure:

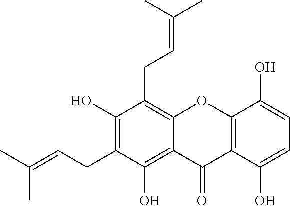

Rutin is the glycoside related to quercetin and rutinose, and is also known as rutoside, phytomelin, sophorin, biturin, eldrin, bitrutin forte, rutin trihydrate globularicitrin, violaquercitrin, quercetin-3-rutinoside, vitamin P, and sophorin. It can be found in a number of plants including, for example, cranberries, mulberries, buckwheat, asparagus, lemons, limes, oranges, grapefruit. Rutin generally has the following chemical structure:

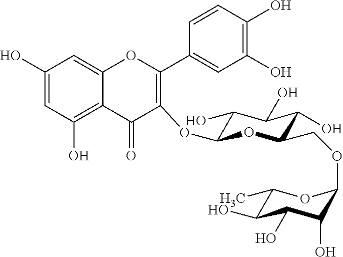

Quercetin is also known as Sophoretin, Meletin, Quercetine, Xanthaurine, Quercetol, Quercitin, Quertine, and Flavin meletin. It can be utilized to help prevent and treat several medical conditions including, for example, cancer, cataracts, bronchitis, allergies, inflammation, prostatitis, asthma, and high blood pressure. Quercetin generally has the following chemical structure:

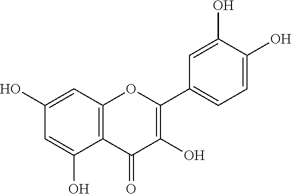

Ginko Bilboa extract compounds can include ginko flavinoids. Kaempferol, also sometimes known as also known as Swartziol, Kempferol, Populnetin, Trifolintin, Rhamnolutin, Rhamnolutein, Pelargidenolon, and Robigenin, is one constituent in ginkgo flavonoids. Recent studies indicate kaempferol may have antitumor activities, effectively inhibit pancreatic cancer cell proliferation and induce cancer cell apoptosis. Kaempferol may also have clinical applications as adjuvant therapy in the treatment of pancreatic cancer. Kaempferol generally has the following chemical structure:

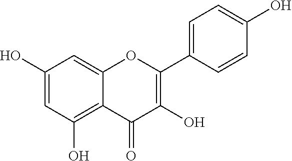

Ocimum sanctum is also known as holy basil. Some extract compounds derived from ocimum sanctum include Oleanolic acid, Ursolic acid, Rosmarinic acid, Eugenol, Carvacrol, Linalool, and f3-caryophyllene.

Rosemary extract compounds can include carnosic acid, rosmarinic acid, camphor, caffeic acid, ursolic acid, betulinic acid, rosmaridiphenol, and rosmanol carnosic acid. Carnosic acid, for example, may shield the brain from free radicals, lowering the risk of strokes and neurodegenerative diseases like Alzheimer's and Lou Gehrig's disease.

Blueberry extract compounds can include phytochemicals that can exhibit a variety of anticarcinogenic properties, including, for example, to inhibit triple-negative breast tumor growth.

Withania somnifera Dunal extract compounds can be derived from the roots or elaves of the Withania somnifera Dunal plant, also known as Ashwagandha, or Indian ginseng. Withania somnifera Dunal extract compounds can contain bioactive withanolides that can inhibit cyclooxygenase enzymes, lipid peroxidation, and proliferation of tumor cells. The plant Withania somnifera Dunal is widely used in the Ayurvedic system of medicine to treat tumors, inflammation, arthritis, asthma, and hypertension.

Rhodiola extract compounds can be derived from Rhodiola Root rosea, also known as Golden Root, Roseroot, or Aaron's Rod, and can be effective for improving mood and alleviating depression associated with cancer.

Schizandra berry extract compounds can be derived from Schizandra berry, also known as Schizandra chinensis. Chemical constituents that can be included in Schizandra berry extract compounds include, for example, schizandrin, deoxyschizandrin, schisanhenol, schizandrol, sesquicarene, citral, stigmasterol, vitamin C, and vitamin E. Schizandra berry has been used in traditional Chinese medicine to support a healthy functioning endocrine system and digestive system, to support normal liver function, and as a convalescent tonic herb when the kidney system is involved.

Aloe vera extract compounds can include emodin, acemannan, aloeride, and di(2-ethylhexyl)phthalate (DEHP). These compounds may have immunomodulating and anticancer effects. Aloe vera compounds have also been utilized in the treatment of constipation, treat burns, heal wounds, treat psoriasis, frostbite, ulcerative colitis and diabetes.

The preferred amount of each potential secondary component can vary depending upon the component. For example, a secondary component can include up to about 1 g of turmeric extract compounds, up to about 1 g of beta-carotene, up to about 1 g of saw palmetto extract compounds, up to about 5 g of fermented noni juice compounds, up to about 5 g of L-ascorbic acid, up to about 500 mg of Solanum Dulcamara extract compounds, up to about 500 mg of Celastrol, up to about 500 mg of Garcinia mangostana L. (Guttiferae) pericarp extract compounds, up to about 1 g of rutin, up to about 1 g of quercetin, up to about 1 g of ginko bilboa extract compounds, up to about 1 g of ocimum sanctum extract compounds, up to about 1 g of rosemary extract compounds, up to about 1 g of blueberry extract compounds, up to about 1 g of Withania somnifera Dunal extract compounds, up to about 1 g of Rhodiola extract compounds, up to about 1 g of Schizandra berry extract compounds, or up to about 5 g of aloe vera extract compounds.

Some generally preferred secondary components include tumeric extract compounds, fermented noni juice compounds, and L-ascorbic acid. When L-ascorbic acid is utilized, it can preferably be present in a compositional dosage in an amount from about from about 5 mg to about 3,000 mg. Further, the L-ascorbic acid can preferably be present in the form of at least one mineral salt. Additionally, other secondary components can be preferred for treatment of certain medical conditions. For example, saw palmetto is a preferred secondary component in compositional dosages for treating or preventing prostate cancer.

Some examples of compositional dosages include a mixture of secondary components. Preferably, the mixture of secondary components can be present in an amount of up to about 5 g, and the weight ratio of at least one tocotrienol to the mixture of secondary components can be from about 1:10 to about 10:1. In one example, a mixture of secondary components can include at least turmeric extract compounds and fermented noni juice compounds.

In some examples, compositional dosages can be contained within a carrier or dispersion medium. In some examples, the at least one tocotrienol and the at least one secondary component can be a suspension or a colloid in a dispersion medium. The dispersion medium can include at least one substance such as sesame oil, olive oil, canola oil, vegetable oil, corn oil, walnut oil, mineral oil, orange oil, almond oil, rice bran oil, peanut oil, coconut oil, palm oil extracts, animal fat, lecithin, glycerin, and combinations thereof. Preferably, the dispersion medium is tocopherol-free, or substantially tocopherol-free as discussed above.

### Example 1

A prostate health formulation can be prepared as a compositional dosage that may have efficacy in treating prostatic diseases such as prostate cancer and prostatic hyperplasia. The compositional dosage can be administered in one or more units, where each unit includes two gelatin capsules (gel caps) to be administered orally five times per day on a daily basis, where each gelatin capsule contains 200 mg gamma tocotrienol, 75 mg delta tocotrienol, 100 mg turmeric extract, and 200 mg saw palmetto extract, which can be prepared in a dispersion medium of tocopherol-free sesame oil

### Example 2

A breast health formulation can be prepared as a compositional dosage that may have efficacy as a preventive for breast cancer. The compositional dosage can include two gelatin capsules (gel caps) to be administered orally on a daily basis, where each gelatin capsule contains 100 mg gamma tocotrienol, 25 mg delta tocotrienol, 100 mg turmeric extract, and 50 mg L-ascorbic acid as calcium ascorbate, which can be prepared in a dispersion medium of tocopherol-free sesame oil. The compositional dosage can also include 100 mls of fermented noni juice and 100 mls of mangosteen juice, which can be administered orally separate from the gelatin capsules.

From the foregoing, it will be appreciated that although specific examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

What is claimed is:

1. A compositional dosage for daily administration to a subject, the compositional dosage comprising:
at least one tocotrienol in an amount from about 10 mg to about 2 g, the at least one tocotrienol being selected from the group consisting of gamma tocotrienol or a derivative of gamma tocotrienol, delta tocotrienol or a derivative of delta tocotrienol, beta tocotrienol or a derivative of beta tocotrienol, and combinations thereof;
at least one secondary component, the at least one secondary component being present in an amount of up to about 5 g;
wherein the weight ratio of at least one tocotrienol to the at least one secondary component is from about 1:10 to about 10:1.

2. The compositional dosage of claim 1, wherein the at least one tocotrienol is present in an amount from about 50 mg to about 1 g.

3. The compositional dosage of claim 1, wherein the at least one secondary component is derived from a natural source.

4. The compositional dosage of claim 1, wherein the at least one secondary component is selected from the group consisting of turmeric extract compounds, beta-carotene, saw palmetto extract compounds, fermented noni juice compounds, L-ascorbic acid, Solanum Dulcamara extract compounds, Celastrol, Garcinia mangostana L. (Guttiferae) pericarp extract compounds, rutin, quercetin, ginko bilboa extract compounds, ocimum sanctum extract compounds, rosemary extract compounds, blueberry extract compounds, Withania somnifera Dunal extract compounds, Rhodiola extract compounds, Schizandra berry extract compounds, aloe vera extract compounds, and combinations thereof.

5. The compositional dosage of claim 4, wherein the at least one secondary component is selected from the group consisting of up to about 1 g of turmeric extract compounds, up to about 1 g of beta-carotene, up to about 1 g of saw palmetto extract compounds, up to about 5 g of fermented noni juice compounds, up to about 5 g of L-ascorbic acid, up to about 500 mg of Solanum Dulcamara extract compounds, up to about 500 mg of Celastrol, up to about 500 mg of Garcinia mangostana L. (Guttiferae) pericarp extract compounds, up to about 1 g of rutin, up to about 1 g of quercetin, up to about 1 g of ginko bilboa extract compounds, up to about 1 g of ocimum sanctum extract compounds, up to about 1 g of rosemary extract compounds, up to about 1 g of blueberry extract compounds, up to about 1 g of Withania somnifera Dunal extract compounds, up to about 1 g of Rhodiola extract compounds, up to about 1 g of Schizandra berry extract compounds, up to about 5 g of aloe vera extract compounds, and combinations thereof.

6. The compositional dosage of claim 1, further comprising from about 5 mg to about 3,000 mg of L-ascorbic acid.

7. The compositional dosage of claim 4, wherein the L-ascorbic acid is present in the form of at least one mineral salt.

8. The compositional dosage of claim 1, wherein the compositional dosage comprises a suspension or a colloid of the at least one tocotrienol and the at least one secondary component in a dispersion medium.

9. The compositional dosage of claim 8, wherein the dispersion medium comprises at least one substance selected from the group consisting of sesame oil, olive oil, canola oil, vegetable oil, corn oil, walnut oil, mineral oil, orange oil, almond oil, rice bran oil, peanut oil, coconut oil, palm oil extracts, animal fat, lecithin, glycerin, and combinations thereof.

10. The compositional dosage of claim 8, wherein the dispersion medium is substantially tocopherol-free.

11. The compositional dosage of claim 1, wherein the compositional dosage is substantially tocopherol-free.

12. The compositional dosage of claim 1, wherein the at least one tocotrienol comprises gamma tocotrienol or a derivative of gamma tocotrienol and delta tocotrienol or a derivative of delta tocotrienol.

13. The compositional dosage of claim 1, wherein the compositional dosage comprises:
gamma tocotrienol in an amount from about 200 mg to about 400 mg;
delta tocotrienol in an amount form about 50 mg to about 150 mg;
tumeric extract in an amount from about 150 mg to about 250 mg; and
a dispersion medium comprising at least one substance selected from the group consisting of sesame oil, olive oil, canola oil, vegetable oil, corn oil, walnut oil, mineral oil, orange oil, almond oil, rice bran oil, peanut oil, coconut oil, palm oil extracts, animal fat, lecithin, glycerin, and combinations thereof.

14. The compositional dosage of claim 1, wherein the compositional dosage is contained in a delivery system adapted for oral administration, the delivery system being selected from the group consisting of capsules, tablets, liquid solutions, suspensions, and elixirs.

15. The compositional dosage of claim 1, wherein the compositional dosage comprises a therapeutically effective amount of the at least one tocotrienol and the at least one secondary component to prevent or treat at least one medical condition selected from the group consisting of benign tissue growths, pre-cancerous lesions, cancer, inflammations, viral infections, bacterial infections, fungal infections, parasitic infections, impaired bodily function, cell damage, and tissue damage.

16. A method of delivering a compositional dosage to a subject on a daily basis, the method comprising:
providing a compositional dosage, the compositional dosage comprising at least one tocotrienol in an amount from about 10 mg to about 2 g, the at least one tocotrienol being selected from the group consisting of gamma tocotrienol or a derivative of gamma tocotrienol, delta tocotrienol or a derivative of delta tocotrienol, beta tocotrienol or a derivative of beta tocotrienol, and combinations thereof, and at least one secondary component in an amount of up to about 5 g, wherein the weight ratio of at least one tocotrienol to the at least one secondary component is from about 1:10 to about 10:1; and
administering the compositional dosage to the subject in one or more units.

17. The method of claim 16, wherein the compositional dosage is contained in a delivery system adapted for oral administration, the delivery system being selected from the group consisting of capsules, tablets, liquid solutions, suspensions, and elixirs

18. The method of claim 16, wherein the compositional dosage is substantially tocopherol-free.

19. The method of claim 16, wherein the at least one secondary component is selected from the group consisting of turmeric extract compounds, beta-carotene, saw palmetto extract compounds, fermented noni juice compounds, L-ascorbic acid, Solanum Dulcamara extract compounds, Celastrol, Garcinia mangostana L. (Guttiferae) pericarp extract compounds, rutin, quercetin, ginko bilboa extract compounds, ocimum sanctum extract compounds, rosemary extract compounds, blueberry extract compounds, Withania somnifera Dunal extract compounds, Rhodiola extract compounds, Schizandra berry extract compounds, aloe vera extract compounds, and combinations thereof.

20. The method of claim 16, wherein the at least one tocotrienol comprises gamma tocotrienol or a derivative of gamma tocotrienol and delta tocotrienol or a derivative of delta tocotrienol, and the at least one secondary compound comprises turmeric extract.
